(19) United States
(12) Patent Application Publication
(10) Pub. No.: US 2024/0170103 A1
(43) Pub. Date: May 23, 2024

(54) PHASELESS AUXILIARY-FIELD QUANTUM MONTE CARLO WITH DIRECT PRODUCT MULTI-SLATER DETERMINANTS TRIAL

(71) Applicants: Lemon Inc., Grand Cayman (KY); Beijing Youzhuju Network Technology Co., Ltd., Pinggu District, Beijing (CN)

(72) Inventors: Hung Pham, Los Angeles, CA (US); Dingshun Lv, Beijing (CN)

(21) Appl. No.: 18/185,760

(22) Filed: Mar. 17, 2023

(51) Int. Cl. G16C 10/00; G06N 10/70

(52) U.S. Cl. CPC G16C 10/00; G06N 10/70

(57) ABSTRACT

Example embodiments of the present disclosure relate to a solution for ph-AFQMC with direct product multi-Slater determinants trial. Multiple active spaces of a molecular system may be obtained and multiple coefficient tensors may be determined respectively. A composite coefficient tensor may be determined based on a tensor product of the multiple coefficient tensors of the multiple active spaces, and a trial wave function may be further determined based on the composite coefficient tensor and a cutoff value. As such, the multiple coefficient tensors for the multiple active spaces may be determined, thus the computation can be reduced. Additionally, since a cutoff value is used, the composite coefficient tensor is a sparse tensor and the number of Slater determinants may be reduced. Further, the determined trial wave function may be further used in a ph-AFQMC algorithm, and a balance between accuracy and efficiency may be achieved.

FIELD

Example embodiments of the present disclosure generally relate to the field of quantum chemistry, and in particular, to a method, and a non-transitory computer readable storage medium and a device for phaseless auxiliary-field quantum Monte Carlo (ph-AFQMC) with direct product multi-Slater determinants trial.

### BACKGROUND

Quantum chemistry aims to finding a solution to ground-state many-electron problems on classical digital computers. As one of the variants of Quantum Monte Carlo (QMC) which has been a widely recognized technique in quantum chemistry in recent years, phaseless auxiliary-field quantum Monte Carlo (ph-AFQMC) has been known for its balance between accuracy and efficiency. It is known that the selection of a trial wave function significantly impacts the accuracy and scalability of ph-AFQMC. Therefore, how to select the trial wave function is needed to be further studied to improve the accuracy and scalability of ph-AFQMC.

### SUMMARY

In general, example embodiments of the present disclosure provide a solution for ph-AFQMC with direct product multi-Slater determinants trial.

In a first aspect, there is provided a method. The method comprises: obtaining a plurality of active spaces of a molecular system; determining a plurality of coefficient tensors for the plurality of active spaces respectively; determining a composite coefficient tensor based on a tensor product of the plurality of coefficient tensors of the plurality of active spaces; and determining a trial wave function based on the composite coefficient tensor and a cutoff value.

In a second aspect, there is provided a device. The device comprises: at least one processor; and at least one memory storing instructions that, when executed by the at least one processor, cause the device at least to: obtain a plurality of active spaces of a molecular system; determine a plurality of coefficient tensors for the plurality of active spaces respectively; determine a composite coefficient tensor based on a tensor product of the plurality of coefficient tensors of the plurality of active spaces; and determine a trial wave function based on the composite coefficient tensor and a cutoff value.

In a third aspect, there is provided a non-transitory computer readable storage medium. The non-transitory computer readable storage medium has computer executable instructions stored thereon, the instructions, when executed by a device, cause the device to perform: obtaining a plurality of active spaces of a molecular system; determining a plurality of coefficient tensors for the plurality of active spaces respectively; determining a composite coefficient tensor based on a tensor product of the plurality of coefficient tensors of the plurality of active spaces; and determining a trial wave function based on the composite coefficient tensor and a cutoff value.

In a fourth aspect, there is provided a computer program comprising instructions, which, when executed by an apparatus, cause the apparatus at least to perform the method in the first aspect.

It is to be understood that the summary section is not intended to identify key or essential features of embodiments of the present disclosure, nor is it intended to be used to limit the scope of the present disclosure. Other features of the present disclosure will become easily comprehensible through the following description.

### BRIEF DESCRIPTION OF THE DRAWINGS

Some example embodiments will now be described with reference to the accompanying drawings, in which:

FIG. 1 illustrates an example flow of method in accordance with some example embodiments of the present disclosure;

FIG. 2 illustrates an example flow in accordance with some example embodiments of the present disclosure;

FIG. 3A illustrates a comparison result of potential energy curves generated by the solution of the present disclosure and various known methods;

FIG. 3B illustrates a comparison result of the deviations of different methodologies from MRCI-Q;

FIG. 3C illustrates an example of number of determinants as a function of N—N distance for two cutoff values, i.e., ε=0.005 and ε=10⁻⁸, for both CASSCF and LASSCF wave functions in accordance with some example embodiments of the present disclosure; and

FIG. 4 illustrates a simplified block diagram of a device that is suitable for implementing some example embodiments of the present disclosure.

Throughout the drawings, the same or similar reference numerals represent the same or similar elements.

### DETAILED DESCRIPTION

Principle of the present disclosure will now be described with reference to some example embodiments. It is to be understood that these embodiments are described only for the purpose of illustration and help those skilled in the art to understand and implement the present disclosure, without suggesting any limitation as to the scope of the disclosure. The disclosure described herein can be implemented in various manners other than the ones described below.

In the following description and claims, unless defined otherwise, all technical and scientific terms used herein have the same meaning as commonly understood by one of ordinary skills in the art to which this disclosure belongs.

References in the present disclosure to “one embodiment,” “an embodiment,” “an example embodiment,” and the like indicate that the embodiment described may include a particular feature, structure, or characteristic, but it is not necessary that every embodiment includes the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

It shall be understood that although the terms “first” and “second” etc. may be used herein to describe various elements, these elements should not be limited by these terms. These terms are only used to distinguish one element from another. For example, a first element could be termed a second element, and similarly, a second element could be termed a first element, without departing from the scope of example embodiments. As used herein, the term “and/or” includes any and all combinations of one or more of the listed terms.

The terminology used herein is for the purpose of describing particular embodiments only and is not intended to be limiting of example embodiments. As used herein, the singular forms “a”, “an” and “the” are intended to include the plural forms as well, unless the context clearly indicates otherwise. It will be further understood that the terms “comprises”, “comprising”, “has”, “having”, “includes” and/or “including”, when used herein, specify the presence of stated features, elements, and/or components etc., but do not preclude the presence or addition of one or more other features, elements, components and/or combinations thereof. As used herein, “at least one of the following: <a list of two or more elements>” and “at least one of <a list of two or more elements>” and similar wording, where the list of two or more elements are joined by “and” or “or”, mean at least any one of the elements, or at least any two or more of the elements, or at least all the elements.

Some terms being used in the present disclosure and their explanations are listed below in Table 1.

TABLE 1

| | Term | Explanation |
|---|---|---|
| 1 | Active Space | In multi-reference methods, the active space refers to the subset |
| | | of molecular orbitals and electrons that are included in the |
| | | calculation, and are allowed to change their occupation in |
| | | different electronic configurations. |
| 2 | Active Space | A computational method in quantum chemistry used to |
| | Decomposition (ASD) | represent the electronic structure of a molecular system by |
| | | partitioning it into smaller active spaces. |
| 3 | Basis Functions | Mathematical functions used in quantum mechanics to |
| | | represent the wave function. |
| 4 | Configuration | A mathematical expression used to approximate the electronic |
| | Interaction Wave | wave function of a many-electron system by constructing it as a |
| | Function | linear combination of multiple configurations. |
| 5 | Coupled Cluster (CC) | A quantum mechanical method used to study the electronic |
| | Theory | structure of molecules by approximating the wave function as a |
| | | sum of single, double, and higher excitations of electrons, and |
| | | representing the wave function using the exponential operator. |
| 6 | Exchange-Correlation | A mathematical function that describes the relationship |
| | Functional | between the electron density and the exchange and correlation |
| | | energy of a many-electron system. |
| 7 | Fermionic Phase | A challenge encountered in quantum Monte Carlo simulations |
| | Problem | of many-electron systems, where the sign of the wave function |
| | | can become negative, leading to destructive interference of |
| | | different paths and rendering the calculation unfeasible or |
| | | inaccurate. |
| 8 | Ground State | The lowest-energy state of a quantum system, such as an atom, |
| | | molecule, or solid. |
| 9 | Imaginary-Time | Imaginary-time propagation is a numerical method used in |
| | Propagation | quantum Monte Carlo simulations and other computational |
| | | techniques to simulate the time-evolution of quantum systems. |
| 10 | Kohn-Sham Density | A widely used method in computational chemistry and |
| | Functional Theory | materials science that utilizes electron density to describe the |
| | (KS-DFT) | electronic structure of molecules and solids. |
| 11 | Localized-Active | A computational method in quantum chemistry used to |
| | Space Self-Consistent | represent the electronic structure of a molecular system by |
| | Field | partitioning it into smaller active spaces similar to ASD. |
| 12 | Low-Rank | A method used in quantum chemistry to represent a many-body |
| | Approximation (LRA) | wave function as a sum of a few rank-one basis states. |
| | Using Rank-One Basis | |
| | States | |
| 13 | Many-Electron | Many-electron problems are systems with multiple interacting |
| | Problems | electrons, which are difficult to solve using traditional |
| | | methods, and are important in understanding the properties of |
| | | materials and chemical reactions. |
| 14 | Matrix Product States | A type of tensor network-based method used in condensed |
| | (MPS) | matter physics and quantum information science, which |
| | | represent the wave function of a many-body system as a |
| | | product of local tensors. |
| 15 | Multi-Slater | A mathematical expression used in quantum mechanics to |
| | Determinant (MSD) | represent the wave function of a many-electron system as a |
| | | linear combination of multiple Slater determinants. |
| 16 | Multireference | A quantum chemical method used to calculate the electronic |
| | Configuration | structure and properties of molecules, by approximating the |
| | Interaction With | many-electron wave function as a linear combination of |
| | Davidson Correction | multiple Slater determinants, and using a perturbative |
| | (MRCI + Q) | correction to improve the accuracy of the calculation. |
| 17 | Multireference Method | A computational approach in quantum chemistry used to |
| | | accurately describe molecules or materials with complex |
| | | electronic structure, by accounting for all possible electronic |
| | | configurations, including those with more than one open-shell |
| | | or near-degenerate states, which cannot be adequately |
| | | described by single-reference methods. |
| 18 | N-Electron Valence | A multi-reference method used in quantum chemistry to |
| | State Perturbation | accurately describe the electronic structure and properties of |
| | Theory (NEVPT2) | molecules, by expanding the wave function in terms of |
| | | N-electron valence states, which are characterized by the |
| | | occupation of valence orbitals, and treating the interaction |
| | | between these states as a perturbation. |
| 19 | Perturbation Theory | A method used in quantum mechanics to approximate the |
| | (PT) | behavior of a quantum system when subjected to a small |
| | | perturbation, allowing the calculation of its energy, wave |
| | | function. |
| 20 | Phaseless | A type of quantum Monte Carlo method. |
| | Auxiliary-Field | |
| | Quantum Monte Carlo | |
| | (ph-AFQMC) | |
| 21 | Potential Energy Curve | A graph that shows how the energy of a molecular system |
| | (PEC) | changes as a function of one or more internal coordinates, such |
| | | as bond length or angle. |
| 22 | Quantum Chemistry | Quantum chemistry is a branch of chemistry that applies |
| | | principles of quantum mechanics to study the behavior of |
| | | atoms and molecules, including their structure, bonding, and |
| | | reactivity. |
| 23 | Quantum Monte Carlo | A method in computational physics and chemistry that uses |
| | (QMC) | Monte Carlo sampling to solve the Schrodinger equation and |
| | | approximate the electronic properties of molecules and |
| | | materials. |
| 24 | Selected Configuration | A type of configuration interaction method used in quantum |
| | Interaction (sCI) | chemistry to accurately calculate the electronic structure and |
| | | properties of molecules, by including a subset of important |
| | | configurations in the wave function expansion, rather than the |
| | | complete set, allowing for efficient and accurate calculations. |
| 25 | Slater Determinants | A mathematical expression used to represent the wave function |
| | (SD) | of a many-electron system. |
| 26 | Strong Correlation | In quantum mechanics, strong correlation refers to a situation |
| | | where the behavior of a many-electron system cannot be |
| | | accurately described using single-reference methods. |
| 27 | Strongly Correlated | A physical system in which the behavior of its constituents |
| | System | (e.g., electrons, atoms, or molecules) cannot be accurately |
| | | described using traditional methods that assume weak |
| | | interactions or a separability of particles. |
| 28 | Tensor Network-Based | A family of mathematical techniques used in quantum |
| | Methods | many-body physics and chemistry, which represent the wave |
| | | function of a many-body system as a tensor network and allow |
| | | for efficient and accurate computation of quantum properties. |
| 29 | Trial Wave Function | A proposed approximation to the true ground state wave |
| | | function of a many-body system, and is used to guide the |
| | | sampling of configurations in the Monte Carlo calculation. |
| 30 | Unrestricted | A type of coupled-cluster method in which single and double |
| | Coupled-Cluster with | excitations are treated explicitly and triple is treated in a |
| | Single Double And | perturbative manner. |
| | Perturbative Triple | |
| | (UCCSD(T)) | |
| 31 | Wave Function | A mathematical function that describes the quantum state of a |
| | | particle or a system of particles. |

The main challenge in quantum chemistry lies in finding a computationally efficient and accurate solution to ground state many-electron problems on classical digital computers. The difficulty stems from the fact that the most accurate methods tend to be the most computationally expensive. In cases where the number of electrons is large, alternative approaches of lower precision, such as KS-DFT, should be utilized. KS-DFT, known for its low computational cost and reasonable accuracy, has become the method of choice in practical applications of computational chemistry, physics, materials science, and computational biology. However, KS-DFT generally lacks predictive power, as its accuracy often depends on the choice of exchange-correlation functional, which may not be known a priori for the system of interest, and its inability to handle systems with strong electron correlation.

State-of-the-art methods beyond KS-DFT, including coupled cluster theory, perturbation theory, tensor network-based methods, and Quantum Monte Carlo, have good polynomial scaling, but they remain computationally expensive, even for medium-sized systems. Furthermore, these methods offer a trade-off between accuracy and efficiency, but their ability to systematically improve and approach the exact solution is essential for their usefulness in chemical simulations. The discovery of a computationally efficient and accurate method is crucial for advancing the development of new drugs, functional materials, and for gaining deeper insights into chemical processes that play a significant role in technological progress.

QMC has become a widely recognized technique in quantum chemistry in recent years. Among its variants, ph-AFQMC has been known for its balance between accuracy and efficiency. The selection of the trial wave function, however, significantly impacts the accuracy and scalability of ph-AFQMC. The utilization of a trial wave function is essential for ensuring statistical efficiency and mitigating the renowned fermionic phase (or sign) problem.

The ph-AFQMC, when combined with MSD trials generated from a multi-reference method, denoted as MSD-AFQMC, exhibits excellent accuracy for systems with strong electron correlation, while maintaining a computationally manageable scaling. Despite its demonstrated advantages, the application of ph-AFQMC with MSD trials can still pose a considerable computational challenge, with a typical scaling of O(N_{c}·N⁴), where N_{c} refers to the number of Slater determinants or configurations, and N represents the number of basis functions. It is noted that the number of Slater determinants (N_{c}) increases rapidly with the size of the active space, thereby limiting its application to medium-size chemical systems with small active spaces.

Example embodiments of the present disclosure provide a solution for ph-AFQMC with direct product multi-Slater determinants trial. Multiple active spaces of a molecular system may be obtained and multiple coefficient tensors for the multiple active spaces may be determined respectively. A composite coefficient tensor may be determined based on a tensor product of the multiple coefficient tensors of the multiple active spaces, and a trial wave function may be further determined based on the composite coefficient tensor and a cutoff value. According to embodiments of the present disclosure, the multiple coefficient tensors for the multiple active spaces may be determined, thus the computation can be reduced. Additionally, since a cutoff value is used, the composite coefficient tensor is a sparse tensor and the number of Slater determinants may be reduced. As such, the determined trial wave function may be further used in a ph-AFQMC algorithm, and a balance between accuracy and efficiency may be achieved. Principles and some example embodiments of the present disclosure will be described in detail below with reference to the accompanying drawings.

It is noted that the term “tensor” in the present disclosure may be replaced as a vector or a matrix in some cases, and the number of elements in a tensor may correspond to the number of SDs.

The ph-AFQMC algorithm is also called as a “projector” QMC algorithm because it projects towards the ground state from an initial wave function which has a non-zero overlap with the true, exact ground state of the system. A theoretical foundation behind any projector QMC methods may be represented as Equation (1), with the ground state being an asymptotic solution of the imaginary-time Schrödinger equation.

$$\vert {Ψ}_{0} 〉 = {lim}_{τ \to ∞} {e}^{- τ \hat{H}} \vert {Φ}_{0} 〉 \qquad ( Equation 1 )$$

The Equation (1) may be regarded as a process of imaginary-time propagation of an initial wave function. |Φ₀ [symbol] is an initial wave function that has some overlap with |Ψ₀ [symbol] (which is the unknown ideal ground state), τ is the imaginary time, and Ĥ is the Hamiltonian. For example, the Hamiltonian Ĥ may be an ab initio Hamiltonian.

The ph-AFQMC algorithm employs an importance sampling technique that is based on a trial wave function (denoted as |Ψ_{T} [symbol] ) during the open-ended random walk process. The global wave function at a given time τ, which is denoted as |Ψ(τ) [symbol] ) can be written as a weighted statistical sum over N walkers |ψᵢ(τ) [symbol] :

$$\vert Ψ ( τ ) 〉 = {\sum }_{i = 1}^{{N}_{ω}} {ω}_{i} ( τ ) \frac{\vert {ψ}_{i} ( τ ) 〉}{〈 {Ψ}_{T} \vert {ψ}_{i} ( τ ) 〉} \qquad ( Equation 2 )$$

where ωᵢ(τ) is the walker weight.

The local energy (represented as E_{loc}) for each walker at a given time τ can be estimated using the mixed estimator, and represented as Equation (3). Accordingly, the global energy (represented as E₀) at any time r can be estimated in a statistical manner, and represented as Equation (4):

$${E}_{loc} = \frac{〈 {Ψ}_{T} \vert \hat{H} \vert {ψ}_{i} ( τ ) 〉}{〈 {Ψ}_{T} \vert {ψ}_{i} ( τ ) 〉} \qquad ( Equation 3 )$$

$${E}_{0} ( τ ) = \frac{{\sum }_{i = 1}^{{N}_{ω}} {ω}_{i} ( τ ) {E}_{loc}}{{\sum }_{i = 1}^{{N}_{ω}} {ω}_{i} ( τ )} \qquad ( Equation 4 )$$

When assessing the computational cost of a ph-AFQMC calculation, there are three key components to consider: (1) Determining the overlap between the trial wave function and the walker, [symbol] Ψ_{T}|ψᵢ(τ) [symbol] ; (2) Calculating the Green's function to calculate the force bias; and (3) Estimating the local energy. Table 2 displays the computational complexity associated with evaluating the local energy in ph-AFQMC calculations using MSD trials, where N denotes the number of basis functions, and N_{c} denotes the number of Slater determinants.

TABLE 2

| | Cost per sample |
|---|---|
| Overlap | [symbol] (N_{c} + N³) |
| Green's function | [symbol] (N_{c} + N³) |
| Local energy | [symbol] (N_{c}N⁴) |
| | [symbol] (N_{c}N + N⁴) |
| | [symbol] (N_{c}N² + N³) |

According to Table 2, it is shown that the computational complexity is associated with the number of basis functions and the number of Slater determinants. As such, the complexity is particularly pronounced for molecular systems that are both large in size and require a large number of Slater determinants. To mitigate this complexity, reducing the number of Slater determinants (N_{c}) in the MSD trials can significantly reduce the computational cost of ph-AFQMC for extended molecules. Therefore, improving the compactness of MSD trials for ph-AFQMC calculations through reducing the number of important Slater determinants N_{c}, would greatly expand the potential of ph-AFQMC for the simulation of chemical systems with complex electronic structures.

It is well recognized that there is a significant degree of sparsity in the configuration interaction wave function for a number of important real-world applications, such as the exciton properties in molecular crystals and noncovalent interactions in biological systems. This sparsity can be taken into account when generating the trial for ph-AFQMC calculations to reduce computational cost. Several quantum chemical techniques have been developed for ab initio simulations that make use of this sparsity, including selected configuration interaction (sCI) and matrix product states (MPS) methods. While these techniques can be applied to a wide range of problems, they are often developed as generic tools and do not necessarily benefit from a deep understanding of the system of interest using. However, for the simulation of chemical systems at a large scale, more customized and problem-specific methods may be more practical, even if they sacrifice some generality and black-box nature. In other words, a tailored solution may be more effective for a specific problem, just as the right tool for the job is important in any field.

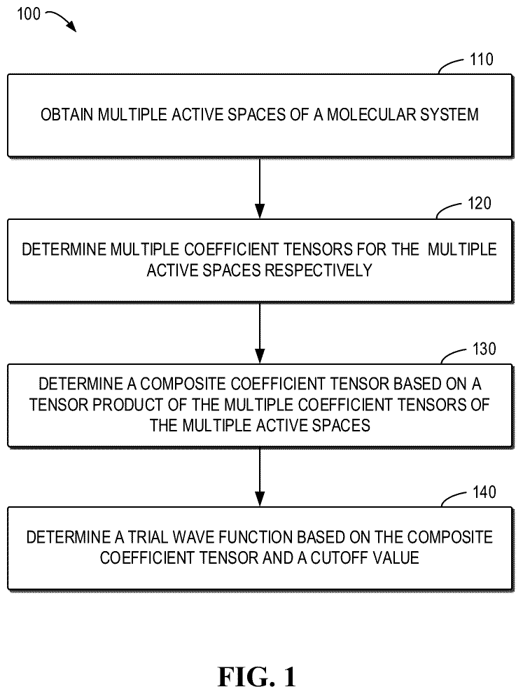

FIG. 1 illustrates an example flow of method 100 in accordance with some example embodiments of the present disclosure. At block 110, multiple active spaces of a molecular system are obtained. At block 120, multiple coefficient tensors for the multiple active spaces are determined respectively. At block 130, a composite coefficient tensor is determined based on a tensor product of the multiple coefficient tensors of the multiple active spaces. At block 140, a trial wave function is determined based on the composite coefficient tensor and a cutoff value.

The method 100 can be applied to a molecular system. For example, the molecular system may include a system with a larger molecule, where the molecule includes multiple fragments or species. For example, the molecular system may include a system with multiple molecules, where the multiple molecules are weakly interacted. For example, the molecular system may include a crystal with multiple different unit cells, where the unit cells are weakly coupled with each other. It is to be understood that the method may also be applied to other molecular system which will not be listed herein.

In some example embodiments, the multiple active spaces are weakly coupled active spaces. In some examples, the molecular system may be decomposed into multiple active spaces based on chemical bounding between multiple subsystems of the molecular system. For example, the multiple subsystems may be the multiple fragments or species of the molecular system. For example, the multiple subsystems may be the multiple molecules of the molecular system. For example, the multiple subsystems may be the multiple different unit cells of the molecular system. In some examples, if two active spaces are separated by a single bond or non-covalent interactions such as van der Waals bond, this may suggests a possible division of the active spaces.

In some example embodiments, a validation of the division of the multiple active spaces may be made. In some examples, complete active space self-consistent field (CASSCF) wave functions may be used to validate the division of the active spaces.

In some example embodiments, for each of the multiple active spaces, respective coefficient tensors may be determined at block 120. Specifically, for a specific active space, a corresponding coefficient tensor may be determined as the coefficient matrix of a configuration interaction (CI) expansion for the specific active space. In some examples, the coefficient tensor may also be called as a CI coefficient tensor with CI coefficients.

In some example embodiments, the composite coefficient tensor determined at block 130 may be a tensor product of the multiple coefficient tensors of the multiple active spaces. Since the direct product approximation exhibits inherent sparsity, the composite coefficient tensor may be a sparse tensor. In other words, it allows an elimination of numerous configurations with near-zero coefficients from the CI expansion. As such, the number of the elements in the composite coefficient tensor may be reduced, and thus the number of SD may be reduced.

The trial wave function may be further determined at block 140 by a tensor product of multiple basis states associated with multiple active spaces. In some examples, the multiple basis states of the multiple active spaces may also be called as multiple monomer wave functions.

The multiple basis states may be determined by using one of some known algorithms, such as an algorithm of active space decomposition (ASD), an algorithm of low-rank approximation (LRA) using rank-one basis states, or an algorithm of localized-active space self-consistent field (LASSCF). In some examples, the multiple monomer wave functions may be constructed and optimized, by utilizing local orbitals as basis states. For example, the algorithm of ASD uses CASSCF and its occupation-restricted variants for orbital optimization. For example, the algorithm of LASSCF utilizes a modified density matrix embedding theory (DMET) algorithm based on an embedding formalism. For example, in the algorithm of LRA, the monomer wave functions are optimized variationally using a method similar to the matrix product states approach.

The composite coefficient tensor determined at block 130 may be used in the trial wave function, where a cutoff value may be further applied to the composite coefficient tensor. In some examples, a rank of the composite coefficient tensor may be greater than (or equal to) the cutoff value. In some examples, the cutoff value may be predefined or may be input by a user. For example, an indication of the cutoff value which is input by the user may be received.

As such, the amount of computation for the trial wave function may be reduced further, and a requirement of memory may be lower.

In some example embodiments, the trial wave function determined at block 140 may be further used to determine an ideal ground state of the molecular system, e.g., by using a ph-AFQMC algorithm. For example, the Equation (1) shown above may be used, where |Φ₀ [symbol] in Equation (1) may be replaced by the trial wave function determined at block 140.

According to the embodiments of the present disclosure, a direct-product wave function can be combined with ph-AFQMC, and the solution may be used for strongly correlated systems, that is, for studying systems that exhibit strong correlation and multiple separable active spaces. Specifically, the construction of the multi-Slater determinant trial from a direct-product wave function is not burdened by an exponential scaling, unlike conventional methods where the configuration coefficient tensor is often dense. This decrease in computational difficulty stems from a fact that the direct-product wave function can be created by combining smaller, easier-to-manage component (monomer) wave functions. Further, the inherent locality of the direct-product MSD (DP-MSD) trial enables the elimination of numerous insignificant configurations for the simulation, as a result of the inherent locality of the component wave function around the component fragments. Additionally, each monomer wave function may be constructed in parallel so as to speed up the process. It is to be understood that the construction of the monomer wave function(s) may be achieved by any suitable method, such as an MPS method.

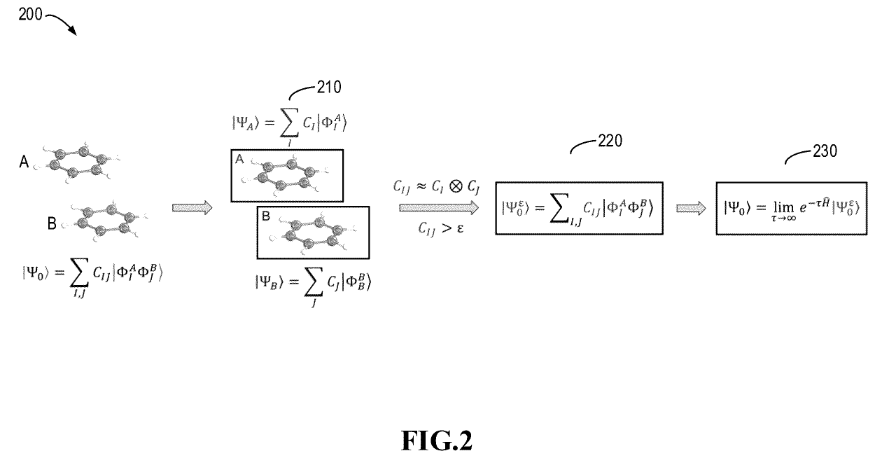

FIG. 2 illustrates an example flow 200 in accordance with some example embodiments of the present disclosure. It is assumed that there are two distinct and weakly coupled active spaces A and B within a system without a loss of generality. Thus, the ground state wave function |Ψ₀ [symbol] can be described by a direct-product form of configuration interaction (CI) expansion:

|Ψ₀ [symbol] =Σ_{I,J}C_{IJ}|Φ_{I}^{A} [symbol] ⊗|Φ_{J}^{B} [symbol] =Σ_{I,J}C_{IJ}|Φ_{I}^{A}Φ_{J}^{B} [symbol] (Equation 5)

where |Φ_{I}^{A} [symbol] and |Φ_{J}^{B} [symbol] denote the many-electron basis states, commonly be referred to as configurations, for active spaces A and B, respectively. C_{IJ} is the coefficient tensor of the CI expansion.

It is understood that the Equation (5) refers to a direct product wave function, which can be generated for ab initio Hamiltonians, by an algorithm, such as active space decomposition (ASD), low-rank approximation (LRA) using rank-one basis states, or localized-active space self-consistent field (LASSCF). Depending on the chosen direct-product wave function, the resulting method is referred to as ASD-AFQMC, LRA-AFQMC, or LAS-AFQMC, respectively. It is to be understood that they all fall under the larger umbrella of Direct-Product State Trial ph-AFQMC (DP-MSD-AFQMC). This notation follows the common convention in quantum chemistry of a “static then dynamic” treatment for electron correlation, which is conveniently decomposed into dynamic and static components.

Electron correlation is commonly divided into two types: static and dynamic. Static correlation arises when there exist multiple electronic configurations with the same energy, and it can be accounted for using a multi-configurational wave function where several Slater determinants are linearly combined to form the wave functions. Dynamic correlation occurs when electrons move and interact with each other, and it can be described by including higher-order excitations in the wave function using perturbation theory. While the separation between static and dynamic correlation is not strict, it is useful in developing new methods to capture a full electron correlation. In quantum chemistry, a multi-reference method can be used to recover static correlation, then a perturbation theory is used to capture the remaining dynamic correlation, that is, a static-then-dynamic approach of treating electron correlation. Although the distinction between dynamic and static correlation within the ph-AFQMC framework is not always clear, this convention is adopted for convenience. The results can be gradually improved by improving the correlation captured in the DP-MSP trial and the total energy can be systematically converged to an exact ground-state energy.

The above CI expansion in Equation (5) for the composite system implicitly embodies the requirement for the Pauli anti-symmetry of fermions. It is worth mentioning that the number of configurations grows exponentially with the size of the combined active space, making direct diagonalization of the Hamiltonian infeasible for larger systems due to prohibitive computational demands. In this regard, the present disclosure presents a solution that the above ground state wave function is computed by diagonalizing the Hamiltonian without explicitly constructing the direct-product basis state |Φ_{I}^{A}Φ_{J}^{B} [symbol] . For example, at 210 as shown in FIG. 2, the Hamiltonians corresponding to active spaces A and B can be diagonalized independently and expressed as:

|Ψ_{A} [symbol] =Σ_{I}C_{I}|Φ_{I}^{A} [symbol] , |Ψ_{B} [symbol] =Σ_{J}C_{J}|Φ_{B}^{B} [symbol] (Equation 6)

where C_{I} and C_{J} are the coefficient tensor of the CI expansion for active spaces A and B, respectively. In other words, C_{I} and C_{J} are computed at 210 in FIG. 2. This leads to an important result that the number of required intermediates in computing the wave function only scales linearly with respect to the number of configurations in each active space calculation.

Regarding the memory requirements, while the storage needed for each CI tensor in each active space increases exponentially with the size of active space A or B, the total storage cost for the combined system is simply the sum of these two costs. Consequently, the present disclosure presents a contrast to directly diagonalizing the total wave function, where the computational cost swiftly becomes infeasible due to the exponential increase.

Accordingly, the composite CI coefficient tensor (C_{IJ}) can be estimated from a tensor product of the monomer CI tensors:

C_{IJ}≈C_{I}⊗C_{J} (Equation 7)

where ⊗ represents a tensor product.

The tensor C_{IJ} within the direct-product approximation of the wave function exhibits inherent sparsity, which allows the elimination of numerous configurations with near-zero coefficients from the CI expansion.

In some example embodiments, a cutoff value (represented as ε) may be defined and input by a user. For example, the user-defined cutoff value ε may be set for the magnitude of the coefficient tensor C_{IJ}.

During practical calculations, the cutoff value may be gradually decreased to incorporate more configurations and achieve convergence of the truncated trial energy towards the energy obtained from the CI expansion trial that utilizes all available configurations. By balancing computational feasibility with the pursuit of convergence in accuracy, the present disclosure seeks to achieve a compromise between these two objectives. As shown at 220 in FIG. 2, the resulting wave function can be represented by the following equation, which defines a direct-product multi-Slater determinant trial, |Ψ₀^{ε} [symbol] :

|Ψ₀ [symbol] ≈|Ψ₀^{ε} [symbol] ^{=Σ}_{I,J}C_{IJ}|Φ_{I}^{A}Φ_{J}^{B} [symbol] , such that |C_{IJ}|>ε(Equation 8)

In some example embodiments, the trial wave function |Ψ₀^{ε} [symbol] as shown in Equation (8) may be called as a DP-MSD trial wave function, which may be used for determining an ideal ground state.

It is to be noted that the memory requirement for storing the wave function |Ψ₀^{ε} [symbol] is significantly lower compared to storing all configurations. As such, the embodiments of the present disclosure adopts a truncation procedure similar to that utilized in the sCI method, where the CI expansion of the variational wave function is sorted and truncated to include only the most relevant configurations. However, the sparsity in the sCI method is not effectively utilized, resulting in dense wave functions that are computationally demanding to achieve the desired level of accuracy. In contrast, the inherent locality of the direct-product wave function in the present disclosure enables more efficient computation.

Further referring to FIG. 2, at 230, the ph-AFQMC algorithm may be performed using the DP-MSD trial wave function as in Equation (8).

The present disclosure may be applied in various real-world applications, e.g., a customized solution may be proposed for the applications such as the study of excitonic properties in molecular crystals and noncovalent interactions in biological systems. In some examples, the present disclosure may be used to investigate electron and exciton dynamics between chromophores, which are found in various contexts, from biological photosynthetic complexes to organic semiconductors used in solar energy conversion. In some examples, the present disclosure may be used for the examination of energy and charge transfer between chromophores and the role of noncovalent interactions in these processes. In some examples, the present disclosure may be used to analyze the function of noncovalent interactions in stabilizing the structures of organic and biological molecules, including proteins, DNA, self-assembled aromatic crystals, and host-guest complexes. For example, aromatic-aromatic, aromatic-cation, and aromatic-hydrogen bonding are some of the noncovalent interactions that play a vital role in these contexts. As such, the present disclosure provides insights into the role of noncovalent interactions in a wide range of organic and biological systems, making it relevant to several fields such as biochemistry, materials science, and biophysics.

According to the embodiments of the present disclosure, a computational efficiency of ph-AFQMC utilizing MSD trials may be enhanced. In some embodiments, the trial construction process becomes simplified as the complete active space is decomposed into smaller and manageable active spaces, thereby mitigating the exponential computational cost associated with generating the trial wave function. In some embodiments, the wave function's inherent compactness enables further truncation of configurations, resulting in a significant reduction in the number of configurations in the final trial. In terms of accuracy, the embodiments of the present disclosure preserve the accuracy of ph-AFQMC as long as the system exhibits multi-reference character with separable active spaces.

A numerical simulation is performed on C₂H₆N₄ using 6-31G basis set to validate the accuracy and computational efficiency of the solution proposed in the present disclosure. In the numerical simulation, the LASSCF algorithm is used due to its ease of implementation.

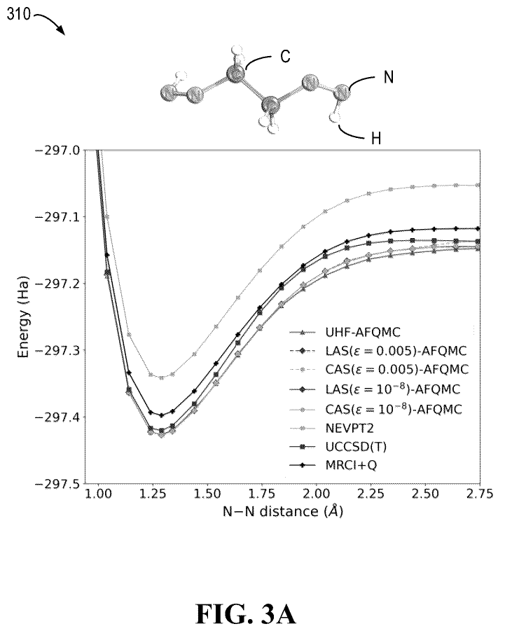

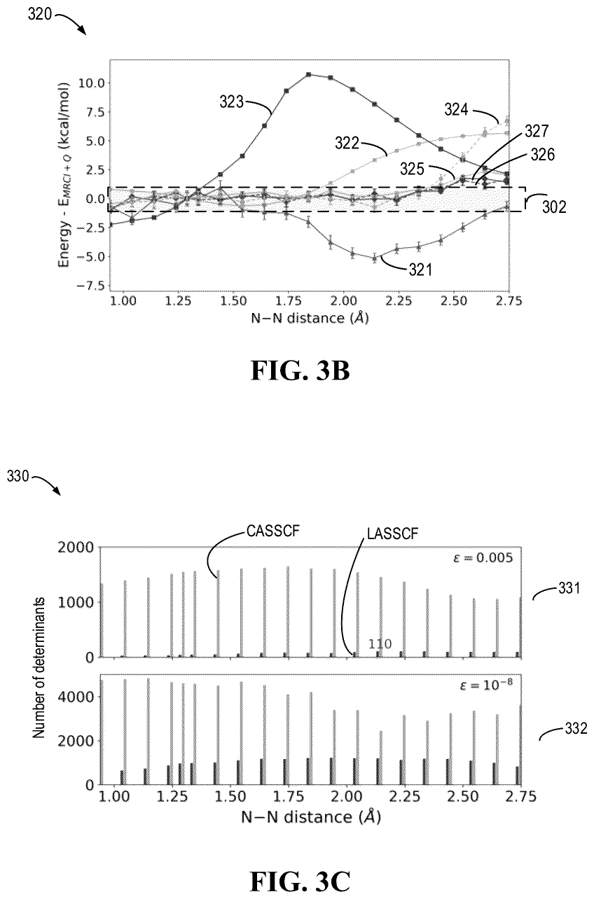

FIG. 3A illustrates a comparison result 310 of potential energy curves generated by different methodologies including the solution of the present disclosure and various known methods. One of the various known methods includes MRCI-Q, which is widely acknowledged as an accurate method and thus it is used as a benchmark in the numerical simulation. FIG. 3B illustrates a comparison result 320 of the deviations of different methodologies from MRCI-Q.

In the numerical simulation of C₂H₆N₄, for example, the two fragments of one larger molecule was divided into two subsystems or monomers. The strength of the electron correlation effects may be enhanced by a dissociation of the N—N bonds, as such it is possible to evaluate the accuracy of our method under varying levels of electron correlation. The LASSCF method is used to divide the large active space with eight electrons in eight orbitals (CAS(8,8)) into two smaller active spaces, each with four electrons in four orbitals (CAS(4,4)).

The methodologies include some known methods, such as the MRCI-Q as a benchmark, the NEVPT2, the UCCSD(T), and the unrestricted Hartree-Fock (UHF)-AFQMC, where the UHF-AFQMC indicates a ph-AFQMC using a single Slater determinant trial. The methodologies also include the solution in the present disclosure with different types of trails: LASSCF (LAS for short) and CASSCF (CAS for short) for two different cutoff values: ε=0.005 and ε=10⁻⁸.

It is to be understood that each potential energy curve (PEC) of the molecule shown in FIG. 3A is obtained by computing its total energy over a range of N—N distances. Accordingly, a difference (i.e., deviation) between a PEC obtained by a method and a PEC obtained by MRCI-Q may be derived and plotted in FIG. 3B, where the deviations are normalized to zero at the equilibrium bond length of 1.29 Å and the chemical accuracy of 1 kcal/mol is indicated by the panel 302. Clearly, the comparison result 320 in FIG. 3B is more suitable since the absolute total energies are irrelevant.

As shown in FIG. 3B, it can be observed from the curve 321 that the UHF-AFQMC is inaccurate in the N—N distance range between approximately 1.50 Å and 2.70 Å. It can be observed that from the curves 322 and 323 that the NEVPT2 and the UCCSD(T) cannot accurately calculate the PEC of this molecule, for example, the NEVPT2 has deviations as large as 5 kcal/mol observed at the strong correlation limit.

However, as shown in FIG. 3B, it can be observed from the curve 324 that the AFQMC using CASSCF as the trial with ε=0.005 as the cutoff is highly accurate up to about 2.30 Å, beyond which its accuracy rapidly declines. And it can be observed from the curve 325 that the AFQMC using CASSCF as the trial with a much tighter cutoff value ε=10⁻⁸, the declined accuracy can be remedied. As shown in FIG. 3B, it can be observed from the curves 326/327 that the AFQMC using LASSCF as the trial are the most accurate result across all bond lengths, regardless of the cutoff values used. Therefore, a compactness of the direct-product multi-Slater determinant trial may be achieved.

FIG. 3C illustrates an example 330 of number of determinants as a function of N—N distance for two cutoff values, i.e., ε=0.005 and ε=10⁻⁸, for both CASSCF and LASSCF wave functions in accordance with some example embodiments of the present disclosure. As shown in FIG. 3C, the graph 331 is associated with a cutoff value ε=0.005 and the graph 332 is associated with a cutoff value ε=10⁻⁸.

It is observed that, compared to the graph 332 when ε=10⁻⁸ is used, the graph 331 shows the number of determinants (such as the number of SDs) in the LASSCF trial when ε=0.005 is used is considerably smaller than that in the CASSCF trial, because of the sparsity of the LASSCF wave function and of the direct-product wave function in general. For example, despite using more than 1000 determinants, the CAS(ε=0.005)-AFQMC method is still unable to calculate the PEC accurately at the strong correlation limit.

It can be concluded that the LAS (ε=0.005)-AFQMC method is the most accurate one among various methodologies in FIG. 3A. For example, as shown in graph 331, at most 110 determinants are required to study the PEC of C₂H₆N₄. The significant reduction in the number of determinants used in LAS-AFQMC supports the notion that this trial, as well as DP-MSD trials more generally, is not only accurate but also highly efficient for systems where the complete active space can be decomposed.

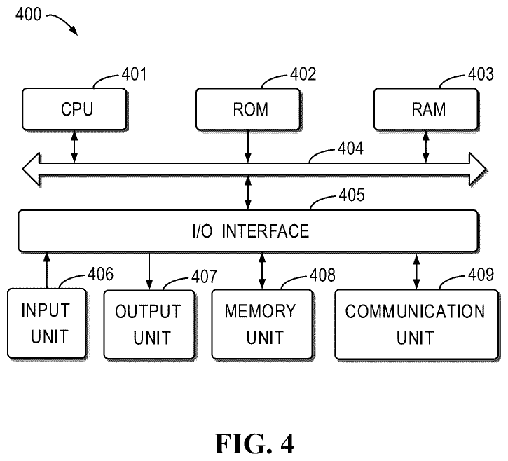

FIG. 4 illustrates a simplified block diagram of a device 400 that is suitable for implementing some example embodiments of the present disclosure. As illustrated therein, the device 400 includes a central processing unit (CPU) 401 that may perform various appropriate actions and processing based on computer program instructions stored in a Read-Only Memory (ROM) 402 or loaded from a memory unit 408 to a Random-Access Memory (RAM) 403. In the RAM 403, there may further store various programs and data needed for operations of the device 400. The CPU 401, ROM 402 and RAM 403 are connected to each other via a bus 404. An input/output (I/O) interface 405 is also connected to the bus 404.

Various components in the device 400 are connected to the I/O interface 405, including: an input unit 406 such as a keyboard, a mouse and the like; an output unit 407 such as various types of displays and loudspeakers, etc.; a memory unit 408 such as a magnetic disk, an optical disk, and etc.; and a communication unit 409 such as a network card, a modem, and a wireless communication transceiver, etc. The communication unit 409 allows the device 400 to exchange information/data with other devices via a computer network such as the Internet and/or various types of telecommunications networks. It is understood that the present disclosure may display, via the output unit 407, real-time dynamic change information of the customer satisfaction, key factor identification information of a group of customers or individual customers subjected to the satisfaction, optimized strategy information, and strategy implementation effect assessment information, etc.

The processing unit 401 may be implemented by one or more processing circuits. The processing unit 401 may be configured to perform various processes and processing described above. For example, in some embodiments, the process described above may be implemented as a computer software program that is tangibly embodied on a machine readable medium, e.g., the memory unit 408. In some embodiments, part or all of the computer program may be loaded and/or mounted onto the device 400 via ROM 402 and/or communication unit 409. When the computer program is loaded to the RAM 403 and executed by the CPU 401, one or more steps of the process as described above may be executed.

It is to be understood that although FIG. 4 is shown as an illustrative device to perform the process or method shown above, the embodiments of the present disclosure may also be implemented at one or more quantum computers, the present disclosure does not limit this aspect.

The present disclosure may be implemented a system, a method and/or a computer program product. The computer program product may comprise a computer-readable storage medium on which computer-readable program instructions for executing various aspects of the present disclosure are loaded.

The computer readable storage medium may be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform various aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processing unit of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the drawings illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It is also to be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Although the present disclosure has been described in languages specific to structural features and/or methodological acts, it is to be understood that the present disclosure defined in the appended claims is not necessarily limited to the specific features or acts described above. Rather, the specific features and acts described above are disclosed as example forms of implementing the claims.

1. A method comprising:
obtaining a plurality of active spaces of a molecular system;
determining a plurality of coefficient tensors for the plurality of active spaces respectively;
determining a composite coefficient tensor based on a tensor product of the plurality of coefficient tensors of the plurality of active spaces; and
determining a trial wave function based on the composite coefficient tensor and a cutoff value.

2. The method of claim 1, further comprising:
determining an ideal ground state based on the trial wave function by using a phaseless auxiliary-field quantum Monte Carlo (ph-AFQMC) algorithm.

3. The method of claim 1, further comprising:
receiving an indication of the cutoff value input by a user.

4. The method of claim 1, wherein a rank of the composite coefficient tensor is not less than the cutoff value.

5. The method of claim 1, wherein the composite coefficient tensor is a direct product of the plurality of coefficient tensors.

6. The method of claim 1, wherein each of the plurality of coefficient tensors is a configuration interaction (CI) expansion of corresponding active space.

7. The method of claim 1, wherein the trial wave function is determined further based on a plurality of basis states for the plurality of active spaces.

8. The method of claim 7, wherein the plurality of basis states for the plurality of active spaces are determined by using one of:
an algorithm of active space decomposition (ASD),
an algorithm of low-rank approximation (LRA) using rank-one basis states, or
an algorithm of localized-active space self-consistent field (LASSCF).

9. The method of claim 1, wherein the composite coefficient tensor is a sparse tensor.

10. The method of claim 1, wherein the plurality of active spaces of the molecular system are determined based on chemical bonding between a plurality of subsystems of the molecular system.

11. A device comprising:
at least one processor; and
at least one memory storing instructions that, when executed by the at least one processor, cause the device at least to:
obtain a plurality of active spaces of a molecular system;
determine a plurality of coefficient tensors for the plurality of active spaces respectively;
determine a composite coefficient tensor based on a tensor product of the plurality of coefficient tensors of the plurality of active spaces; and
determine a trial wave function based on the composite coefficient tensor and a cutoff value.

12. The device of claim 11, wherein the device is further caused to:
determine an ideal ground state based on the trial wave function by using a phaseless auxiliary-field quantum Monte Carlo (ph-AFQMC) algorithm.

13. The device of claim 11, wherein device is further caused to:
receive an indication of the cutoff value input by a user.

14. The device of claim 11, wherein a rank of the composite coefficient tensor is not less than the cutoff value.

15. The device of claim 11, wherein the composite coefficient tensor is a direct product of the plurality of coefficient tensors.

16. The device of claim 11, wherein each of the plurality of coefficient tensors is a configuration interaction (CI) expansion of corresponding active space.

17. The device of claim 11, wherein the trial wave function is determined further based on a plurality of basis states for the plurality of active spaces.

18. The device of claim 17, wherein the plurality of basis states for the plurality of active spaces are determined by using one of:
an algorithm of active space decomposition (ASD),
an algorithm of low-rank approximation (LRA) using rank-one basis states, or
an algorithm of localized-active space self-consistent field (LASSCF).

19. The device of claim 11, wherein the composite coefficient tensor is a sparse tensor.

20. A non-transitory computer readable storage medium having computer executable instructions stored thereon, the instructions, when executed by a device, causing the device to perform:
obtaining a plurality of active spaces of a molecular system;
determining a plurality of coefficient tensors for the plurality of active spaces respectively;
determining a composite coefficient tensor based on a tensor product of the plurality of coefficient tensors of the plurality of active spaces; and
determining a trial wave function based on the composite coefficient tensor and a cutoff value.
